(19) United States
(12) Patent Application Publication
(10) Pub. No.: US 2024/0390576 A1
(43) Pub. Date: Nov. 28, 2024

(54) GLUCOSE MONITOR INJECTION PORT

(71) Applicant: Embecta Corp., Parsippany, NJ (US)

(72) Inventor: Eugene Randel Prais, Milford, NJ (US)

(73) Assignee: Embecta Corp., Parsippany, NJ (US)

(21) Appl. No.: 18/694,103

(22) PCT Filed: Sep. 22, 2021
(86) PCT No.: PCT/US2021/051431

§ 371 Date: Mar. 21, 2024

(51) Int. Cl. A61M 5/142

(52) U.S. Cl. CPC A61M 5/14244; A61M 2005/14252; A61M 2230/201

(57) ABSTRACT

A device for delivering fluid and an inserter in a device for delivering fluid, the device comprising an annular guide configured to engage a pen or a syringe injection needle, a main body enclosed by a base and a cover, and an inserter for expelling the fluid, wherein the inserter includes a glucose monitoring sensor disposed coaxially with the inserter. The inserter comprising a cannula configured to deliver the fluid, and a glucose monitoring sensor disposed coaxially to and surrounding the cannula, wherein the glucose monitoring sensor includes a biosensor layer that monitors glucose in the fluid and provides feedback to the device.

### FIELD OF THE INVENTION

The present invention relates to improvements to existing injection ports and existing glucose monitoring devices.

### BACKGROUND OF THE INVENTION

Some diabetics choose not to subject themselves to multiple direct injections with a syringe. Some reasons include a fear of needles and because they bruise easily. Insulin pumps have become a popular option for diabetics who do not desire multiple daily direct injections with a syringe.

Using an injection port or an insulin pump, for example, the diabetic receives a continuous dosage of insulin from a pump apparatus via an infusion device mounted on their body. Insulin is supplied (e.g., pumped) from the insulin pump through a tube to the infusion device. Infusion devices generally include a cannula mounting in a subcutaneous manner within the flesh of the diabetic. The infusion device includes a channel that transmits insulin from an inlet port to the cannula for being delivered to the subcutaneous tissue layer of the diabetic.

Most conventional infusion devices have an insertion needle that extends through a body of the device and through the cannula. During mounting of the infusion device, the insertion needle pierces a skin of the diabetic and supports the cannula since most cannulas are made from a soft and/or flexible material. Accordingly, the diabetic still must deal with a needle piecing their skin. However, because the infusion device may remain in place for an extended period of time (e.g., typically up to 3 days or more), the diabetic need only deal with one injection type needle over 3 or more days, rather than multiple times per day. This extended period of time between needle insertions is what makes the pump tolerable for many diabetics who have an aversion to being pierced with injection needles.

In patients with diabetes, glucose levels need to be monitored to maintain a healthy balance of glucose in the body. Monitoring blood glucose levels, however, can also require diabetics to endure uncomfortable or unpleasant needle sticks. For example, a diabetic person may also carry a self-monitoring blood glucose (SMBG) monitor, which typically comprises uncomfortable finger pricking methods. Due to the lack of comfort and convenience, a diabetic will normally only measure his or her glucose level two to four times per day. Unfortunately, these time intervals are so far spread apart that the diabetic will likely find out too late, sometimes incurring dangerous side effects, of a hyperglycemic or hypoglycemic condition. In fact, it is not only unlikely that a diabetic will take a timely SMBG value, but additionally the diabetic will not know if their blood glucose value is going up (higher) or down (lower) based on conventional methods.

Alternatively, glucose levels can be monitored by GBP coated sensors such as on-body continuous glucose monitoring (CGM) devices or implantable CGM devices. CGM devices can have a needle or probe that is inserted into the tissue of a user to measure the glucose levels in the surrounding tissue fluid. A transmitter is incorporated into the CGM device to communicate data collected by the CGM device to a separate receiver. Wearing a CGM device requires a needle stick to insert the needle or probe into the tissue of the user, which some diabetics find unpleasant and uncomfortable.

### SUMMARY OF THE INVENTION

In view of the above considerations, an improved injection port and means to measure glucose levels is desired and provided by example embodiments of the present disclosure.

It is an aspect of the present invention to provide a coaxial medication delivery and biosensor system delivering injection port and CGM (continuous glucose monitor) functionality in a combined fluid delivery device. Such a fluid delivery device advantageously integrates the CGM sensor and the delivery cannula to simultaneously measure glucose levels and adjust insulin injection rates based on bodily needs. Specifically, the pancreas in a human body makes a set amount of insulin continuously throughout the day. Basal insulin mimics that process for the human body to absorb slowly and use throughout the day. On the other hand, prandial insulin is taken during mealtime and acts rapidly in the human body to manage the elevation of glucose levels. The fluid delivery device advantageously considers these conditions simultaneously during operation.

It is also aspect of the present invention to provide the glucose monitoring sensor in the fluid delivery device with a continuous self-monitoring of glucose levels to the patient or by a clinician without a needle injection for each measurement. That is, less needle insertions are required which advantageously reduces discomfort. Moreover, body real estate is preserved through the efficiency of the combination of uses in the fluid delivery device. This is important because the user feels more encumbered by the use of multiple devices.

It is another aspect of the present invention to provide the glucose monitoring sensor coaxial to the integrated cannula and surrounding the integrated cannula at its exterior diametric surface. The tubular format of the glucose monitoring sensor is advantageous to the design of the fluid delivery device and distinguishes from a conventional flat ribbon format. The glucose monitoring sensor is advantageously inserted subcutaneously with the integrated cannula. Such a configuration is advantageously easy to apply and easy to use. Further, the fluid delivery device including the integrated cannula and the glucose monitoring sensor can advantageously be worn for up to three days and during all normal activities, including exercising, sleeping and bathing.

The foregoing and/or other aspects of the present invention can be achieved by providing a device for delivering fluid, the device comprising an annular guide configured to engage a pen or a syringe injection needle, a main body enclosed by a base and a cover, and an inserter for expelling the fluid, wherein the inserter includes a glucose monitoring sensor disposed coaxially with the inserter.

The foregoing and/or other aspects of the present invention can also be achieved by providing an inserter in a device for delivering fluid, the inserter comprising a cannula configured to deliver the fluid, and a glucose monitoring sensor disposed coaxially to and surrounding the cannula, wherein the glucose monitoring sensor includes a biosensor layer that monitors glucose in the fluid and provides feedback to the device.

Additional and/or other aspects and advantages of the present invention will be set forth in the description that follows, or will be apparent from the description, or may be learned by practice of the invention.

### BRIEF DESCRIPTION OF THE DRAWINGS

The above aspects and features of the present invention will be more apparent from the description for the exemplary embodiments of the present invention taken with reference to the accompanying drawings, in which:

FIG. 1 illustrates a top perspective view of an exemplary fluid delivery device;

FIG. 2 illustrates a top perspective view of the fluid delivery device of FIG. 1 with a transparent cover over a main body;

FIG. 3 illustrates a right cross-sectional perspective view of the fluid delivery device of FIG. 2; and

FIG. 4 illustrates a left perspective view of the fluid delivery device of FIG. 2 with a reusable electronic module and printed circuit board removed;

FIG. 5 illustrates a left perspective view of the reusable electronic module of FIG. 2;

FIG. 6 illustrates a right cross-sectional perspective view of the fluid delivery device of FIG. 3 with a trocar cover removed;

FIG. 7 illustrates a cross-sectional view of an integrated cannula of FIG. 6;

FIG. 8 illustrates another cross-sectional view of the integrated cannula of FIG. 7; and

FIG. 9 illustrates a block diagram of the fluid delivery device.

### DETAILED DESCRIPTION OF EXEMPLARY EMBODIMENTS

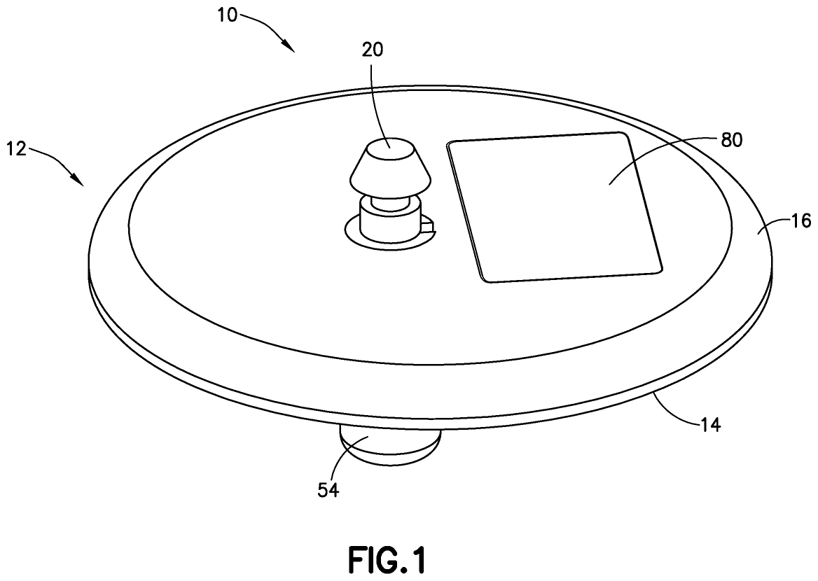

FIG. 1 illustrates a fluid delivery device 10 including a main body 12 enclosed by a base 14 and a cover 16. The fluid delivery device 10 described herein can also be applied to an injection port, for example. The base 14 is configured to be attached to a skin surface of a patient during medication delivery. Adhesive, for example, is typically used to attach the base 14 to the skin surface for approximately three days of use. The cover 16 and the base 14 are preferably silicon. The cover 16 engages the base 14 to surround the contents of the main body 12 and form a soft, flexible on body member. The main body 12 houses the wearable portion of the fluid delivery device 10 and is disposable after use.

The fluid delivery device 10 further includes an annular guide 20. The annular guide 20 is configured to engage a pen, a syringe injection needle, a patch pump or an infusion pump, for example, to provide a sealed, controlled interface for establishing fluid communication. Specifically, during operation, the medicament from the pen or the syringe injection needle can be provided to the fluid delivery device 10 for medication delivery.

The main body 12 carries a reusable electronic module 80 whereby the reusable electronic module 80 is sealed from fluid ingress. The reusable electronic module 80 is disposed in the main body 12 and electrically connects to various electronics in the fluid delivery device 10 for advanced operation and monitoring as further described below. The reusable electronic module 80 is advantageously replaceable for specific medicament delivery configurations and data collection for multiple patients, for example. The reusable electronic module 80 is also visible and accessible with the cover 16 installed whereby the cover 16 including an opening configured to install and remove the reusable electronic module 80.

FIG. 1 also illustrates a trocar cover 54 that covers an inserter assembly 50 comprising an insertion trocar 52, an integrated cannula 56 and a glucose monitoring sensor 58. Specifically, the trocar cover 54 is used to shield the insertion trocar 52 and the integrated cannula 56 prior to and after use to avoid inadvertent injury to the patient. The trocar cover 54 includes an opening on its proximal end for the insertion trocar 52 and integrated cannula 56 to enter. The proximal end of the trocar cover 54 is also configured to engage the base 14 for proper retention. The distal end of the trocar cover 54 is enclosed to prevent exposure of the insertion trocar 52 and the integrated cannula 56. Further details of the inserter assembly 50 are described below.

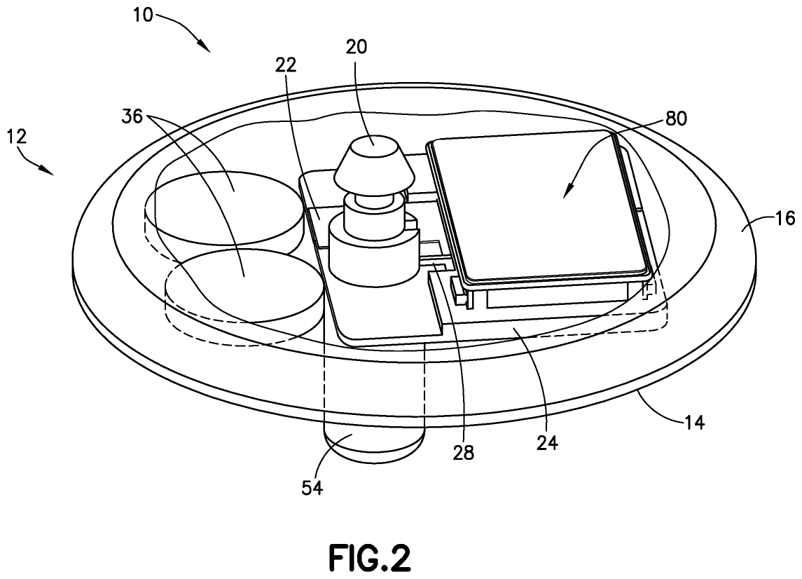

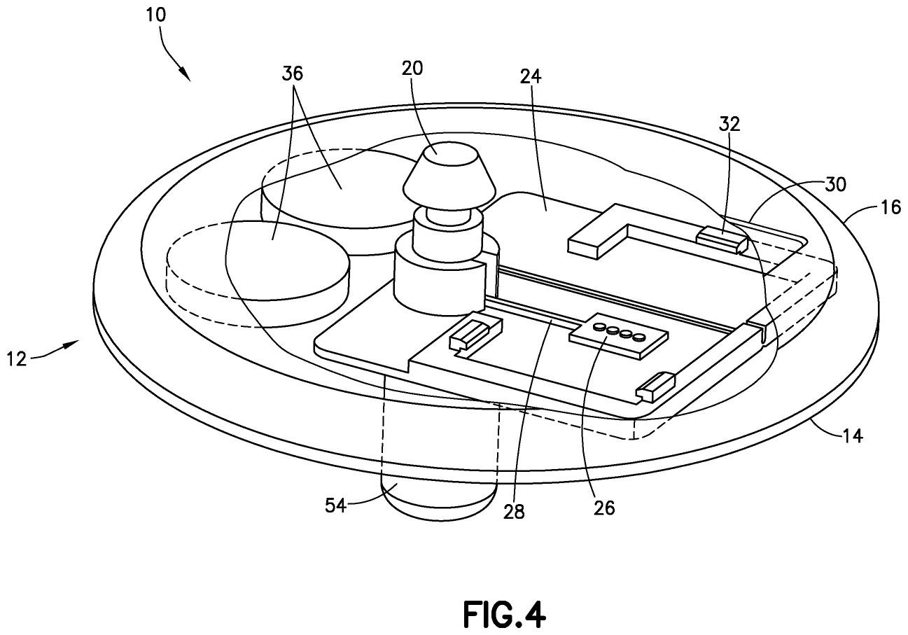

FIGS. 2 and 4 illustrate various components disposed in the main body 12. A flexible frame 24 is used to carry and secure a variety of components. The flexible frame 24 is advantageously flexible to cushion any impact force that any electrical components in the fluid delivery device 10 could experience when the fluid delivery device 10 is dropped or shaken, for example. A printed circuit board 22 is disposed on top of the flexible frame 24. A sensor contact pad 26 is electrically connected to the printed circuit board 22 and is disposed underneath the reusable electronic module 80. Flexible wiring 28 electrically connects the sensor contact pad 26 to the glucose monitoring sensor 58 integrated into the integrated cannula 56 as further described below.

The glucose monitoring sensor 58 advantageously provides a continuous self-monitoring of glucose levels to the patient or by a clinician without a needle injection for each measurement. That is, the fluid delivery device 10 requires less needle insertions which advantageously reduces discomfort. Further details of the glucose monitoring sensor 58 is described below.

The flexible frame 24 includes a recess 30 that is sized to provide a cavity to accept and engage the reusable electronic module 80. The recess 30 includes retention features 32 to secure the reusable electronic module 80 in the recess 30. An exemplary retention feature 32 includes a tab on the reusable electronic module 80 and a locking cavity configured to engage the tab in the recess 30, although other forms of retention arc contemplated herein by one skilled in the art. The main body 12 also includes one or more batteries 36 that is electrically connected (not shown) to the printed circuit board 22 to power the electrical components in the fluid delivery device 10.

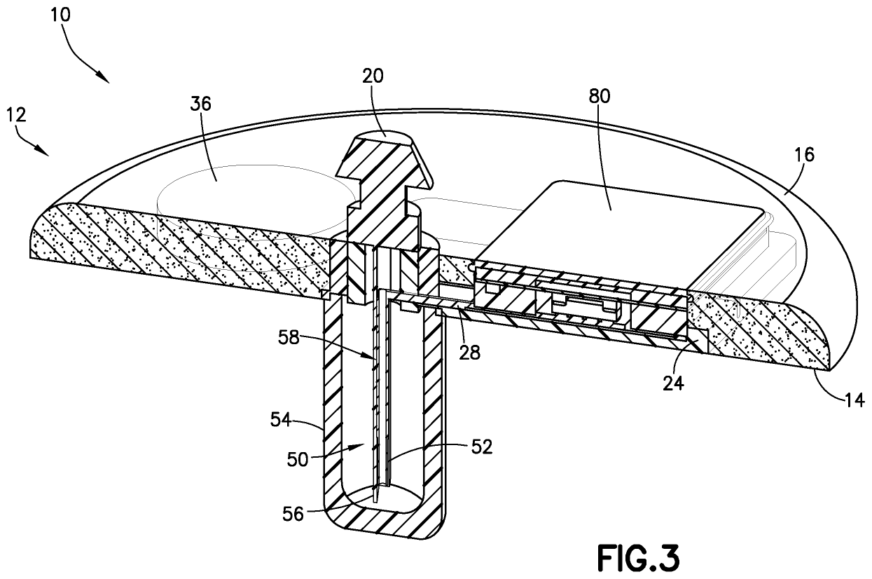

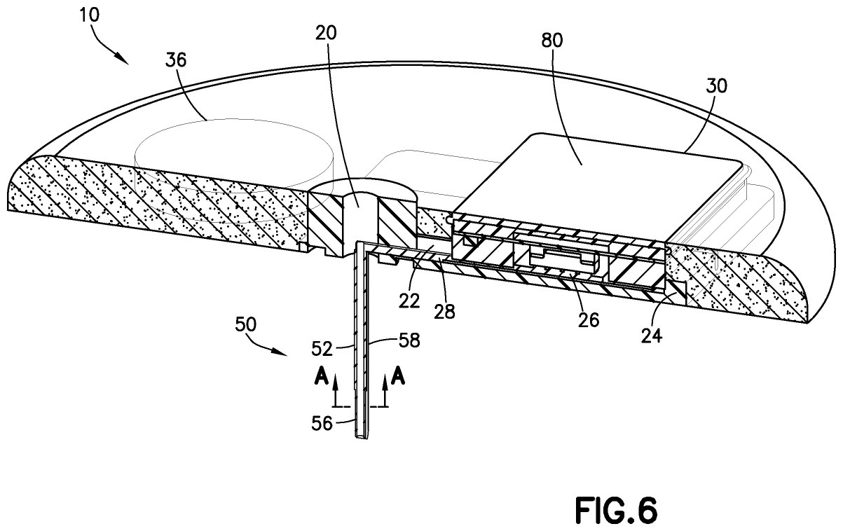

FIGS. 3 and 6 further illustrates a trocar portion subassembly of the inserter assembly 50. The inserter assembly 50 includes the insertion trocar 52 and the trocar cover 54. The insertion trocar 52 includes a hollow cavity surrounding the integrated cannula 56. Alternately, the insertion trocar 52 can be a hollow needle. The distal end of the insertion trocar 52, as understood by one skilled in the art, includes cutting edges that can create an incision in the skin of the patient or a body cavity to remove fluid and/or act as a portal for subsequent placement of other instruments.

The integrated cannula 56 is carried subcutaneously by a U-channel of the insertion trocar 52. The integrated cannula 56 travels with the insertion trocar 52 when inserted into the skin of the patient. Once a desired depth is achieved, the insertion trocar 52 retracts leaving the integrated cannula 56 in an implanted position in the skin of the patient. The insertion trocar 52 can be retracted by a spring mechanism providing a retraction spring force, a lever mechanism or other mechanism or methods as understood by one skilled in the art.

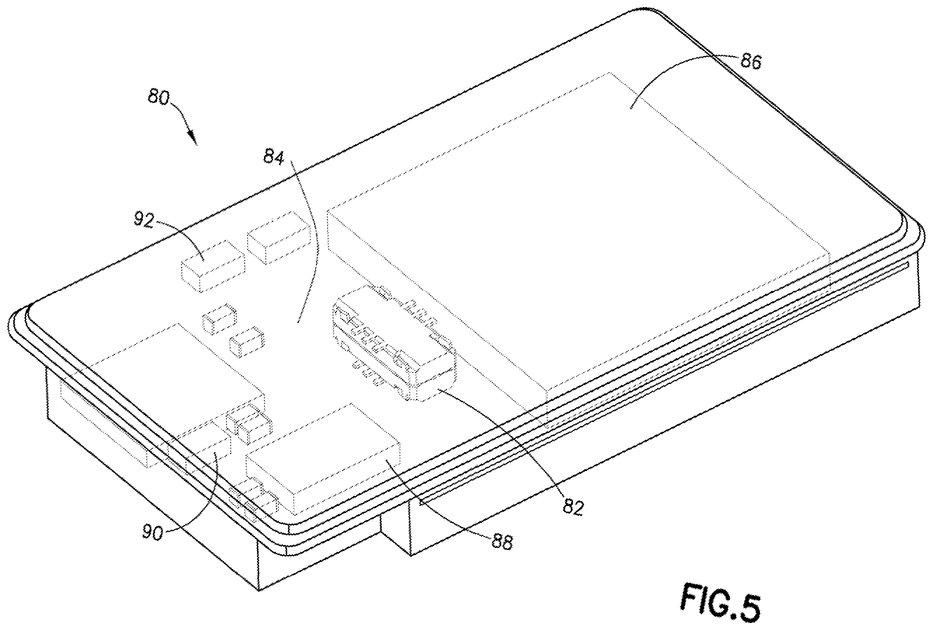

FIG. 5 illustrates the reusable electronic module 80 including a connector 82 and a control panel 84. While the main body 12 and the inserter assembly 50 is a disposable portion of the fluid delivery device 10 and can be disposed after use, the reusable electronic module 80 is a reusable portion of the fluid delivery device 10 and can be transferred to an unused main body 12 and inserter assembly 50 in another fluid delivery device 10 for continued use. The connector 82 is configured to engage the printed circuit board 22 to receive glucose monitoring data and electrical power. A secondary connector is also provided in the reusable electronic module 80 to provide another form of wired communication to another external device, such as a smart device or a laptop. The control panel 84 carries various electrical components as understood by one skilled in the art including a microprocessor 86, a real-time clock 88, a Bluetooth 90 or near field communication 90 and a power management controller 92.

Specifically, the microprocessor 86 includes arithmetic, logic, and control circuitry necessary to perform the functions of the fluid delivery device 10. The real-time clock 88 measures the passage of time to facilitate monitoring and adjusting medicament fluid rates, such as insulin rates, and monitoring glucose levels over time. The Bluetooth® 90 provides wireless communication between the fluid delivery device 10 and, for example, a smart device such as a phone or a tablet. The near field communicator 90 allows for communication between the fluid delivery device 10 and another electronic device in close proximity. A user can simply wave a smart phone, for example, over the near field communicator 90 to exchange data collected by the fluid delivery device 10. The power management controller 92 controls the amount of electrical power consumed by various components in the fluid delivery device 10.

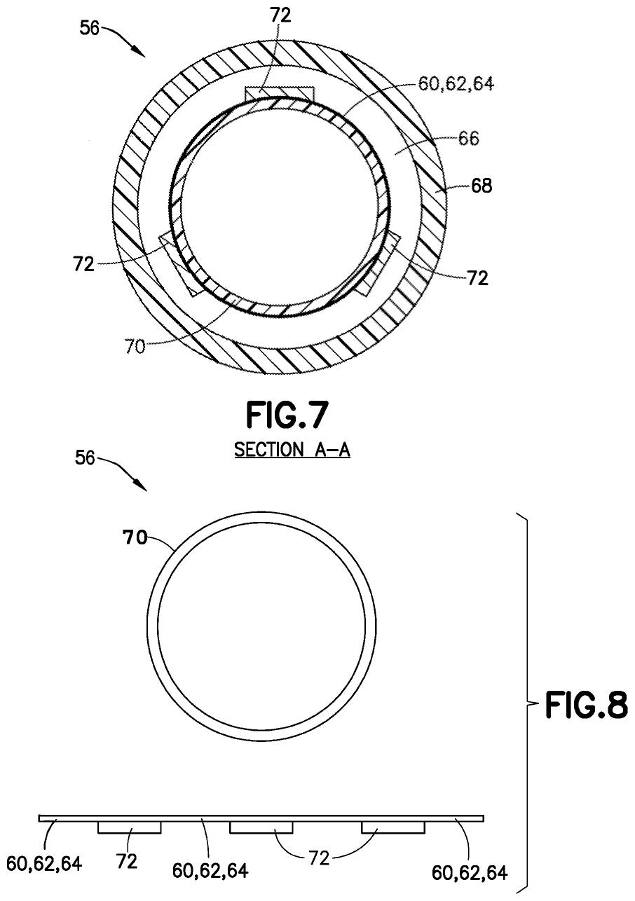

FIGS. 7 and 8 illustrates a cross section of the integrated cannula 56 and the glucose monitoring sensor 58. The integrated cannula 56 is tubular in shape and relatively soft as understood by one skilled in the art to avoid pain to the patient during injection. The integrated cannula 56 is inserted with the insertion trocar 52 to provide a channel for medicament (such as insulin) delivery to a subcutaneous region of the skin. Placement of the fluid delivery device 10 on a patient and insertion of the insertion trocar 52 and integrated cannula 56 into a patient's skin can be accomplished by different methods and devices known in the art (e.g., a disposable or reusable applicator for an injection port).

The glucose monitoring sensor 58 is flexible and does not alter the comfort level of the patient when inserted. The glucose monitoring sensor 58 is advantageously coaxial to the integrated cannula 56 and surrounds the integrated cannula 56 at its exterior diametric surface. The tubular format of the glucose monitoring sensor 58 is advantageous and unique to the design of the fluid delivery device 10 and distinguishes from the conventional flat ribbon format. The glucose monitoring sensor 58 is advantageously inserted subcutaneously with the integrated cannula 56 during operation.

The integrated cannula 56 and the glucose monitoring sensor 58 advantageously cooperates with the control panel 84 to facilitate measuring glucose levels and recording manual injections and/or optionally automatically adjusting insulin injection rates simultaneously, depending on the type of fluid delivery mechanism employed with the device 10 (e.g., pen, syringe, patch pump or infusion pump). Additionally, the glucose monitoring sensor 58 advantageously provides a continuous self-monitoring of glucose levels to the patient or by a clinician without a needle injection for each measurement.

Such a configuration is advantageously easy to apply and easy to use. Further, the fluid delivery device 10 including the integrated cannula 56 and the glucose monitory sensor 58 can advantageously be worn for up to three days and during all normal activities, including exercising, sleeping and bathing. Nevertheless, the glucose monitoring sensor 58 can be extended for up to fourteen days.

The glucose monitoring sensor 58 includes a biosensor layer 60, an enzyme layer 66, a membrane layer 68 a substrate stiffener layer 70 and printed conductive traces 72. The substrate stiffener laver 70 contacts and directly surrounds the outer diameter of the integrated cannula 56. Preferably, the substrate stiffener layer 70 includes a Teflon fluoropolymer, although other materials are contemplated by one skilled in the art. The substrate stiffener layer 70 advantageously stiffens the integrated cannula 56 while preserving the operation of the glucose monitoring sensor 58 and not significantly altering patient comfort during insertion of the integrated cannula 56 and the glucose monitoring sensor 58 into the skin of the patient.

The biosensor layer 60 connects to each of the printed conductive traces 72 and surrounds the substrate stiffening layer 70. Three to four printed conductive traces 72 are printed on the biosensor layer 60 and disposed on an outer diametral surface of the substrate stiffening layer 70. More or less printed conductive traces 72 used in the fluid delivery device 10 are contemplated by one skilled in the art. The printed conductive traces 72 electrically connect to flexible wiring 28 that travels in a proximal direction adjacent to the integrated cannula 56 and terminates at the sensor contact pad 26 in the main body 12.

The biosensor layer 60 includes an electrochemical biosensor layer 62 and a glucose oxidase based sensor layer 64. The uppermost length of the integrated cannula 56 to a distance of approximately six to seven millimeters away from a bottom surface of the base 14 (contacting the skin surface) is preferably where the electrochemical biosensor layer 62 is disposed. This active length for the biosensor layer 60 of approximately six to seven millimeters of depth is typical and commonly used for sensing in an interstitial fluid. A substrate to form the glucose oxidase based sensor layer 64 is preferably constructed by coating the integrated cannula 56 with a polyimide like material to provide a degree of stability and to increase rigidity.

The biosensor layer 60 in cooperation with the printed conductive traces 72 monitor the glucose traveling through the integrated cannula 56 and provide continuous feedback through the flexible wiring 28 and back to the printed circuit board 22 in the main body 12. As illustrated in FIG. 6, the flexible wiring 28 travels in a proximal direction on the outer diameter of the integrated cannula 56 toward the printed circuit board 22 in the main body 12.

FIG. 7 illustrates that the enzyme layer 66 coaxially surrounds the biosensor layer 60 and the printed conductive traces 72. The enzyme layer 66 comprises a conductive polymer layer formed in an electropolymerization process where the conducting polymer is electrochemically deposited on the surface of the sensing electrode with the enzyme trapped in the film.

The membrane layer 68 coaxially surrounds the enzyme layer 66. The membrane layer 68 includes one or more layers that moderates the enzyme reactions in the enzyme layer 66. The membrane layer 68 also seals and keeps the glucose monitoring sensor 58 together while surrounding the integrated cannula 56.

The combination of the integrated cannula 56 and the glucose monitoring sensor 58 is advantageously simple in design while providing the benefits of both. The integrated cannula 56 and glucose monitoring sensor 58 are advantageously and conveniently combined because a base in both a separate injection port and a separate glucose monitoring device are similar in size. Additionally, both devices are used at similar locations on the body. Full functionality of a continuous glucose monitor device includes glucose readings, connectivity, alarms and integrated diabetes management over a wear life of the device. The combined continuous glucose monitor device and injection port as described herein as the fluid delivery device 10 advantageously operates in nearly the same manner as the independent injection port and the independent continuous glucose monitor device.

The pancreas in a human body makes a set amount of insulin continuously throughout the day. Basal insulin mimics that process for the human body to absorb slowly and use throughout the day. On the other hand, prandial insulin is taken during mealtime and acts rapidly in the human body to manage the elevation of glucose levels. The fluid delivery device advantageously considers these conditions simultaneously during operation.

Having one wearable that provides both functions advantageously offers enough combined benefit to boost acceptance of wearables by people with disability, while providing cost savings. Moreover, body real estate is preserved through the efficiency of the combination of uses in the fluid delivery device 10. Finally, the user feels more encumbered and less comfortable by multiple devices.

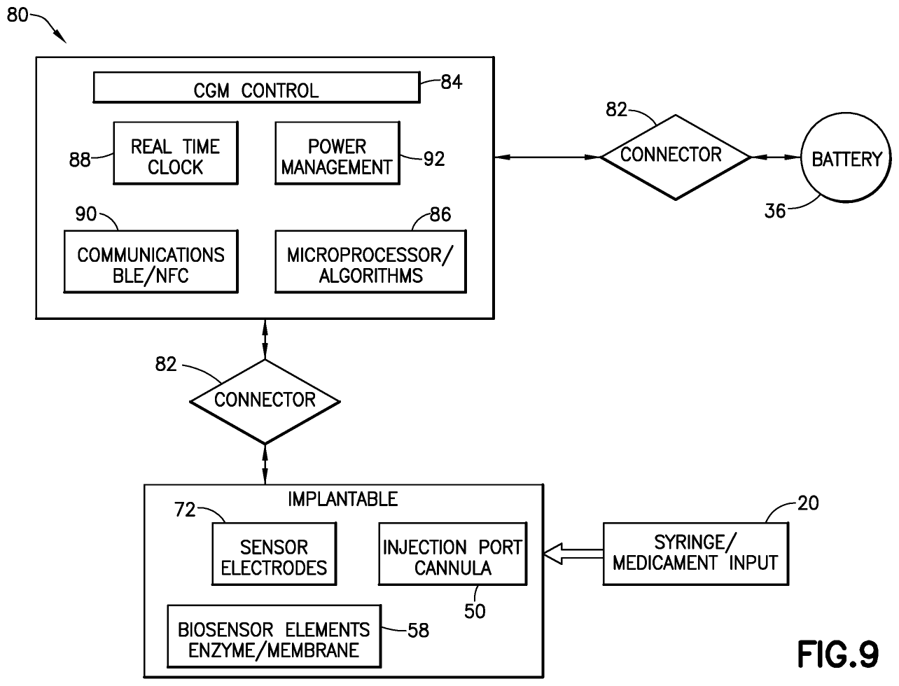

FIG. 9 illustrates a block diagram of all the electrical components of the fluid delivery device 10. The control panel 84 of the reusable electronic module 80 carries the microprocessor 86, real-time clock 88, Bluetooth, or near field communicator 90 and the power management controller 92. The control panel 84 is electrically connected to the printed circuit board 22. The battery 36 is also electrically connected to the printed circuit board 22 to provide electrical power for operation of all the electrical components in the fluid delivery device 10.

The connector 82 also electrically connects the reusable electronic module 80 to the inserter assembly 50. Specifically, the sensor contact pad 26 is electrically connected to the glucose monitoring sensor 58 surrounding the integrated cannula 56 via the flexible wiring 28. The glucose monitoring sensor 58 communicates data to the reusable electronic module 80 via the flexible wiring 28, to the printed circuit board 22 and ultimately to the connector 82. As described above, the annular guide 20 is in fluid communication with the inserter assembly 50 and receives a syringe, a pen or other medicament supply to supply medicament to the fluid delivery device 10.

The foregoing detailed description of the certain exemplary embodiments has been provided for the purpose of explaining the principles of the invention and its practical application, thereby enabling others skilled in the art to understand the invention for various embodiments and with various modifications as are suited to the particular use contemplated. This description is not necessarily intended to be exhaustive or to limit the invention to the precise embodiments disclosed. Any of the embodiments and/or elements disclosed herein may be combined with one another to form various additional embodiments not specifically disclosed, as long as they do not contradict each other. Accordingly, additional embodiments are possible and are intended to be encompassed within this specification and the scope of the invention. It will be understood by one skilled in the art that this disclosure is not limited in its application to the details of construction and the arrangement of components set forth in the above description or illustrated in the drawings. The embodiments herein are capable of other embodiments, and capable of being practiced or carried out in various ways. The specification describes specific examples to accomplish a more general goal that may be accomplished in another way.

Also, it will be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not restricted to physical or mechanical connections or couplings. Further, terms such as up, down, bottom, and top are relative, and are employed to aid illustration, but are not limiting.

Furthermore, as used in this application, the terms “front,” “rear,” “upper,” “lower,” “upwardly,” “downwardly,” and other orientational descriptors are intended to facilitate the description of the exemplary embodiments of the present invention, and are not intended to limit the structure of the exemplary embodiments of the present invention to any particular position or orientation. Terms of degree, such as “substantially” or “approximately” are understood by those of ordinary skill to refer to reasonable ranges outside of the given value, for example, general tolerances associated with manufacturing, assembly, and use of the described embodiments.

The components of the illustrative devices, systems and methods employed in accordance with the illustrated embodiments can be implemented, at least in part, in digital electronic circuitry, analog electronic circuitry, or in computer hardware, firmware, software, or in combinations of them. These components can be implemented, for example, as a computer program product such as a computer program, program code or computer instructions tangibly embodied in an information carrier, or in a machine-readable storage device, for execution by, or to control the operation of, data processing apparatus such as a programmable processor, a computer, or multiple computers.

A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a communication network. Also, functional programs, codes, and code segments for accomplishing the illustrative embodiments can be easily construed as within the scope of claims exemplified by the illustrative embodiments by programmers skilled in the art to which the illustrative embodiments pertain. Method steps associated with the illustrative embodiments can be performed by one or more programmable processors executing a computer program, code or instructions to perform functions (e.g., by operating on input data and/or generating an output). Method steps can also be performed by, and apparatus of the illustrative embodiments can be implemented as, special purpose logic circuitry, e.g., an FPGA (field programmable gate array) or an ASIC (application-specific integrated circuit), for example.

The various illustrative logical blocks, modules, and circuits described in connection with the embodiments disclosed herein may be implemented or performed with a general purpose processor, a digital signal processor (DSP), an ASIC, a FPGA or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example, semiconductor memory devices. e.g., electrically programmable read-only memory or ROM (EPROM), electrically erasable programmable ROM (EEPROM), flash memory devices, and data storage disks (e.g., magnetic disks, internal hard disks, or removable disks, magneto-optical disks, and CD-ROM and DVD-ROM disks). The processor and the memory can be supplemented by, or incorporated in special purpose logic circuitry.

Those of skill in the art would understand that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Those of skill would further appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of claims exemplified by the illustrative embodiments. A software module may reside in random access memory (RAM), flash memory, ROM, EPROM, EEPROM, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. In other words, the processor and the storage medium may reside in an integrated circuit or be implemented as discrete components.

Computer-readable non-transitory media includes all types of computer readable media, including magnetic storage media, optical storage media, flash media and solid state storage media. It should be understood that software can be installed in and sold with a central processing unit (CPU) device. Alternatively, the software can be obtained and loaded into the CPU device, including obtaining the software through physical medium or distribution system, including, for example, from a server owned by the software creator or from a server not owned but used by the software creator. The software can be stored on a server for distribution over the Internet, for example.

The above-presented description and figures are intended by way of example only and are not intended to limit the illustrative embodiments in any way except as set forth in the following claims. It is particularly noted that persons skilled in the art can readily combine the various technical aspects of the various elements of the various illustrative embodiments that have been described above in numerous other ways, all of which are considered to be within the scope of the claims.

1. A device for delivering fluid, the device comprising:
an annular guide configured to engage a pen or a syringe injection needle;
a main body enclosed by a base and a cover; and
an inserter for expelling the fluid; wherein
the inserter includes a glucose monitoring sensor disposed coaxially with the inserter.

2. The device of claim 1, wherein:
the inserter includes a cannula; and
the glucose monitoring sensor surrounds an external diameter of the cannula.

3. The device of claim 2, wherein the glucose monitoring sensor includes a substrate stiffener layer comprising a Teflon fluoropolymer surrounding the cannula.

4. The device of claim 3, wherein:
the glucose monitoring sensor further includes a biosensor layer having an electrochemical biosensor layer and a glucose oxidase based sensor layer; and
the biosensor layer surrounds the substrate stiffener layer.

5. The device of claim 4, further comprising conductive traces electrically connected to the biosensor layer that provides feedback to a printed circuit board in the main body.

6. The device of claim 4 wherein:
the glucose monitoring sensor further includes:
an enzyme layer surrounding the biosensor layer; and
a membrane layer surrounding the enzyme layer.

7. The device of claim 1, further comprising:
an insertion trocar surrounding the glucose monitoring sensor; and
an insertion trocar cover to shield the insertion trocar when not in use.

8. The device of claim 1, wherein the main body includes a frame having a sensor contact pad electrically connected to the glucose monitoring sensor to receive sensor feedback.

9. The device of claim 1, further includes one or more batteries disposed in the main body that are electrically connected to the glucose monitoring sensor.

10. The device of claim 1, wherein the main body includes a frame having retention features configured to secure and release a reusable electronic module.

11. The device of claim 10, wherein the reusable electronic module includes a connector that is configured to engage a printed circuit board in the main body.

12. The device of claim 10, wherein the reusable electronic module includes a control panel having one or more of a microprocessor, a real-time clock, a Bluetooth, a near field communicator and a power management controller.

13. The device of claim 1, wherein the device delivers the fluid by injecting into or through a patient's skin.

14. The device of claim 1, wherein the base is configured to be attached on a patient's skin.

15. The device of claim 1, wherein the annular guide is configured to engage a patch pump or an infusion pump.

16. An inserter in a device for delivering fluid, the inserter comprising:
a cannula configured to deliver the fluid; and
a glucose monitoring sensor disposed coaxially to and surrounding the cannula; wherein
the glucose monitoring sensor includes a biosensor layer that monitors glucose in the fluid and provides feedback to the device.

17. The inserter of claim 16, wherein the device is an injection port.

18. The inserter of claim 16 wherein:
the glucose monitoring sensor further includes:
an enzyme layer surrounding the biosensor layer; and
a membrane layer surrounding the enzyme layer.

19. The inserter of claim 16, wherein the glucose monitoring sensor includes a substrate stiffener layer surrounded by the biosensor layer.

20. The inserter of claim 16, wherein the biosensor layer comprises an electrochemical biosensor layer and a glucose oxidase based sensor layer.
